(19) United States
(12) Patent Application Publication
(10) Pub. No.: US 2013/0317572 A1
(43) Pub. Date: Nov. 28, 2013

(54) LOW-LEVEL LASER THERAPY

(71) Applicant: BOSTON SCIENTIFIC NEUROMODULATION CORPORATION, Valencia, CA (US)

(72) Inventors: Changfang Zhu, Valencia, CA (US); Dongchul Lee, Agua Dulce, CA (US); Kerry Bradley, Glendale, CA (US)

(73) Assignee: BOSTON SCIENTIFIC NEUROMODULATION CORPORATION, Valencia, CA (US)

(21) Appl. No.: 13/902,749

(22) Filed: May 24, 2013

Related U.S. Application Data
(60) Provisional application No. 61/652,093, filed on May 25, 2012.

(51) Int. Cl. A61N 5/06

(52) U.S. Cl. CPC A61N 5/0622
USPC 607/89

(57) ABSTRACT

A method of treating a patient with an ailment using an optical element implanted within the patient, comprising conveying low-level laser energy having a wavelength in the range of 600 nm-2500 nm from the optical element to a neuronal element of the patient, thereby modulating the neuronal element to treat the ailment.

### RELATED APPLICATION DATA

The present application claims the benefit under 35 U.S.C. §119 to U.S. provisional patent application Ser. No. 61/652,093, filed May 25, 2012. The foregoing application is hereby incorporated by reference into the present application in its entirety.

### FIELD OF THE INVENTION

The present invention relates to tissue modulation systems, and more particularly, to a system and method for therapeutically modulating nerve fibers.

### BACKGROUND OF THE INVENTION

Among many techniques attempted for neurostimulation (e.g., electrical, chemical, mechanical, thermal, magnetic, optical, and so forth), electrical stimulation is the standard and most common technique. Implantable electrical stimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) techniques, which directly stimulate the spinal cord tissue of the patient, have long been accepted as a therapeutic modality for the treatment of chronic pain syndromes, and the application of spinal cord stimulation has begun to expand to additional applications, such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, Functional Electrical Stimulation (FES) systems such as the Freehand system by NeuroControl (Cleveland, Ohio) have been applied to restore some functionality to paralyzed extremities in spinal cord injury patients. Occipital Nerve Stimulation (ONS), in which leads are implanted in the tissue over the occipital nerves, has shown promise as a treatment for various headaches, including migraine headaches, cluster headaches, and cervicogenic headaches. In recent investigations, Peripheral Stimulation (PS), which includes Peripheral Nerve Field Stimulation (PNFS) techniques that stimulate nerve tissue directly at the symptomatic site of the disease or disorder (e.g., at the source of pain), and Peripheral Nerve Stimulation (PNS) techniques that directly stimulate bundles of peripheral nerves that may not necessarily be at the symptomatic site of the disease or disorder, has demonstrated efficacy in the treatment of chronic pain syndromes and incontinence, and a number of additional applications are currently under investigation. Vagal Nerve Stimulation (VNS), which directly stimulate the Vagal Nerve, has been shown to treat heart failure, obesity, asthma, diabetes, and constipation.

Each of these implantable stimulation systems typically includes an electrode lead implanted at the desired stimulation site and neurostimulator (e.g., an implantable pulse generator (IPG)) implanted remotely from the stimulation site, but coupled either directly to the electrode lead or indirectly to the electrode lead via a lead extension. Thus, electrical pulses can be delivered from the neurostimulator to the stimulation lead(s) to stimulate or activate a volume of neural tissue. In particular, electrical energy conveyed between at least one cathodic electrode and at least one anodic electrode creates an electrical field, which when strong enough, depolarizes (or “stimulates”) the neurons beyond a threshold level, thereby inducing the firing of action potentials (APs) that propagate along the neural fibers. The stimulation regimen will typically be one that provides stimulation energy to all of the target tissue that must be stimulated in order to provide the therapeutic benefit, yet minimizes the volume of non-target tissue that is stimulated.

The stimulation system may further comprise a handheld remote control (RC) to remotely instruct the neurostimulator to generate electrical stimulation pulses in accordance with selected stimulation parameters. The RC may, itself, be programmed by a technician attending the patient, for example, by using a Clinician's Programmer (CP), which typically includes a general purpose computer, such as a laptop, with a programming software package installed thereon. If the IPG contains a rechargeable battery, the stimulation system may further comprise an external charger capable of transcutaneously recharging the IPG via inductive energy.

Although electrical stimulation energy has been proven to be reliable to treating various ailments, including chronic pain, transcutaneous low-level laser radiation (such as pulsed infrared light) is known to be effective to alleviate pain (see R. Chow, et al., Inhibitory Effects of Laser Irradiation on Peripheral Mammalian Nerves and Relevance to Analgesic Effects: A Systemic Review, Photomedicine and Laser Surgery, 29(6), 365-381 (2011); Synder-Mackler, et al., The Effect of Helium-Neon Laser on Latency of Sensory Nerve, Physical Therapy, 68: 223-225 (1988); G. D. Baxter, et al., Effects of Low Intensity Infrared Laser Irradiation Upon Conduction in the Human Median Nerve In Vivo, Experimental Physiology 79, 227-234 (1994); Toru Kono, et al., Cord Dorsum Potentials Suppressed by Low Power Laser Irradiation on a Peripheral Nerve in the Cat, Journal of Clinical Laser Medicine & Surgery, 11(3), 115-118 (1993)).

Whereas the primary mechanism of electrical stimulation is the external impulse current or voltage stimuli that modulates the voltage gated ion channels on neuronal membrane, thereby modulating the excitability of neuronal element, the recent studies have showed that the mechanism underlying infrared optical stimulation is likely the localized heating converted from the absorbed optical energy that alters the electrical capacitance of the neuronal membrane, thereby causing the depolarization of neuronal elements. Some studies have shown that low-level laser energy at a wavelength of 600-900 nm suppresses the activity of neurons by reducing oxidative stress and disrupting fast axonal transport. In contrast, low-level laser energy at longer wavelengths (e.g., 1500-2000 nm) may enhance the activity of neurons.

Thus, electrical and optical energy modalities activate neurons using different mechanisms and have their own advantages and disadvantages. For example, the primary advantages of electrical neural modulation are it is controllable, reliable, well-implemented, and can be easily miniaturized. However, there are safety concerns with electrical neural modulation due to electro-chemical reaction at the tissue-electrode interface. Furthermore, electrical neural modulation may compromise selectivity due to the spread of current. On the other hand, the primary advantages of optical neural modulation are it can be highly focused and delivered in a non-contact manner. However, the generation of pulsed optical energy for direct neuron stimulation may call for a powerful laser driver and complicated system to provide an optical path, thus making it challenging to use this technique in portable or implantable neuromodulation devices.

There, remains a need for improved optical neural modulation devices and techniques as an alternative to, or as an adjunct to, electrical neural modulation.

### SUMMARY OF THE INVENTION

In accordance with a first aspect of the present inventions, a method of treating a patient with an ailment using an optical element implanted within the patient is provided. The method comprises conveying low-level laser energy having a wavelength in the range of 600 nm-2500 nm from the optical element to a neuronal element of the patient, thereby modulating the neuronal element to treat the ailment. The low-level laser energy may decrease the excitability of the neuronal element (e.g., if the wavelength is in the range of 600 nm-1200 nm, and preferably in the range of 600 nm-900 nm) or increase the excitability of the neuronal element (e.g., if the wavelength is in the range of 1400-2500 nm).

In one method, the neuronal element is a central nervous system (CNS) neuronal element. For example, the CNS neuronal element may be a brain structure, such as one of a thalamus, subthalamic nucleus (STN), globus pallidus, subgenual cingulate cortex, rostral cingulate cortex, ventral striatum, nucleus accumbens, inferior thalamic peduncle, lateral habernula, ventromedial hypothalamus, ventrolateral thalamus, zona incerta, posteroventral globus pallidus pars ilnternus, periventricular gray (PVG), periaqueductal gray (PAG), cerebellum, centromedian nucleus of thalamus, anterior nucleus of thalamus, caudate nucleus, mesial temporal lobe, ventral capsule (VC), ventral striatum (VS), pars interna of the globus internal, and anterior limb of internal capsule, in which case, the ailment to be treated may be one or more of Parkinson's disease, depression, addiction, obesity, tremor, dystonia, pain, epilepsy, obsessive compulsive disorder, and Tourette's syndrome. Or the CNS neuronal element may be a spinal cord, such as one of a dorsal column, ventral column, lateral column, and dorsal horn, in which case, the ailment to be treated may be chronic pain.

In another method, the neuronal element is a peripheral nervous system (PNS) neuronal element. For example, the PNS neuronal element may be a spinal nerve or a dorsal root ganglion (DRG).

In accordance with a second aspect of the present inventions, a neuromodulation lead is provided. The neuromodulation lead comprises an elongated body, at least one electrode carried by the distal end of the elongated body, and at least one optical element carried by the distal end of the elongated body. The electrode(s) is configured for conveying electrical energy capable of modulating a neuronal element, whereas the optical element(s) is configured for conveying low-level laser energy capable of modulating a neuronal element. In an optional embodiment, the neuromodulation lead further comprises a connector carried by the proximal end of the elongated body. The connector configured for being mated to a neuromodulation device to couple the neurostimulation device to the electrode(s) and the optical element(s).

In one embodiment, the neuromodulation lead further comprises at least one optical fiber extending through the elongated body and coupled to the optical element(s). In this case, each of the optical element comprises a lens configured for focusing the low-level laser energy, and may further comprise a mirror configured for directing the low-level laser energy from each of the optical fiber(s) to the respective lens. In another embodiment, each of the optical element(s) comprises a laser light emitting diode (LED).

In still another embodiment, the neuromodulation lead comprises a plurality of optical elements, which may be configured for respectively conveying the low-level laser energy in different radial directions. In this case, the neuromodulation lead further comprises a plurality of electrodes interleaved with the plurality of optical elements. The neuromodulation lead may further comprise a plurality of parallel optical fibers extending through the elongated body and respectively coupled to the plurality of optical elements. Or the neuromodulation lead may further comprise an optical fiber extending through the elongated body and coupled to the plurality of optical elements, in which case, the neuromodulation lead may further comprise at least one beam splitter in series with the optical fiber for coupling optical energy carried by the optical fiber to the plurality of optical elements.

In accordance with a third aspect of the present inventions, a method of treating a patient with an ailment. The method comprises conveying electrical energy to a first neuronal element, thereby modulating the first neuronal element, and conveying low-level laser energy having a wavelength in the range of 600 nm-2500 nm to the first neuronal element, thereby modulating the first neuronal element. At least one of the conveyance of the electrical energy and the conveyance of the low-level laser energy treats the ailment. The low-level laser energy may decrease the excitability of the neuronal element (e.g., if the wavelength is in the range of 600 nm-1200 nm, and preferably in the range of 600 nm-900 nm) or increase the excitability of the neuronal element (e.g., if the wavelength is in the range of 1400-2500 nm).

In one method, the electrical energy is conveyed to a second neuronal element, thereby modulating the second neuronal element to treat the ailment, wherein the modulation of the first neuronal element by the low-level laser energy decreases a side-effect otherwise caused by the modulation of the first neuronal element by the electrical energy. The conveyance of the low-level laser energy decreases the excitability of the first neuronal element. As one example, the first neuronal element is a first dorsal root nerve fiber, and the second neuronal element is a second dorsal root nerve fiber. The first and second dorsal root nerve fibers may be at the same vertebral level. As another example, the first neuronal element may be a dorsal column nerve fiber, and the second neuronal element may be a dorsal root nerve fiber. As still another example, the first neuronal element may be a dorsal root nerve fiber, and the second neuronal element may be a dorsal column nerve fiber.

In another method, the conveyance of the electrical energy and/or the low-level laser energy modulates the first neuronal element to treat the ailment. In one example, the low-level laser energy may be conveyed to a portion of a plurality of neuronal elements that includes the first neuronal element, thereby increasing the excitability of the portion of the plurality of neuronal elements, and the electrical energy may be conveyed to the plurality of neuronal elements that includes the first neuronal element while the excitability of the portion of the plurality of neuronal elements is increased, such that portion of the plurality of neuronal elements is stimulated without stimulating a remaining portion of the plurality of neuronal elements. In another example, the electrical energy may be conveyed to a plurality of neuronal elements that includes the first neuronal element, thereby increasing the excitability of the portion of the plurality of neuronal elements, and the low-level laser energy may be conveyed to a portion of the plurality of neuronal elements that includes the first neuronal element while the excitability of the plurality of the plurality of neuronal elements is increased, such that portion of the plurality of neuronal elements is stimulated without stimulating a remaining portion of the plurality of neuronal elements.

In still another method, the electrical energy is conveyed to a first target site along a plurality of neuronal elements that includes the first neuronal element, thereby decreasing the excitability of the plurality of neuronal elements at the first target site, while the low-level laser energy is conveyed at a second target site to a portion of the plurality of neuronal elements that includes the first neuronal element, thereby increasing the excitability of the portion of the plurality of neuronal elements at the second target site, such that only a portion of action potentials intrinsically generated respectively in the plurality of neuronal elements proximal to the first target site is conveyed along the plurality of neuronal elements distal to the second target site.

In yet another method, the electrical energy is conveyed to a first target site along a plurality of neuronal elements that includes the first neuronal element, thereby increasing the excitability of the plurality of neuronal elements at the first target site, while the low-level laser energy is conveyed at a second target site to a portion of the plurality of neuronal elements that includes the first neuronal element, thereby decreasing the excitability of the portion of the plurality of neuronal elements at the second target site, such that only a portion of action potentials intrinsically generated respectively in the plurality of neuronal elements proximal to the first target site is conveyed along the plurality of neuronal elements distal to the second target site.

In yet another method, the electrical energy is conveyed to the first neuronal element at a first target site, thereby bi-directionally evoking action potentials in the first neuronal element, while the low-level laser energy is conveyed to the first neuronal element at a second target site, thereby decreasing the excitability of the first neuronal element at the second target site, such that the action potentials are blocked at the second target site.

In one method, the first neuronal element is a first nerve fiber of a vagus nerve, in which case, the ailment can be one of heart failure, asthma, diabetes, obesity, intestinal disorder, and constipation. The conveyance of the electrical energy may stimulate the first nerve fiber of the vagus nerve innervating a first anatomical region of the patient, thereby treating the ailment, and the conveyance of the electrical energy may decrease the excitability of a second nerve fiber of the vagus nerve innervating a second anatomical region of the patient. Or, electrical energy may be conveyed at a first nerve fiber of the vagus nerve, thereby bi-directionally evoking action potentials in the first nerve fiber, while the low-level laser energy may be conveyed to the first nerve fiber at a second target site, thereby decreasing the excitability of the first nerve fiber at the second target site, such that the action potentials are blocked at the second target site.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is plan view of one embodiment of a neuromodulation system arranged in accordance with the present inventions;

FIG. 2 is a plan view of a fully implantable modulator (FIM) and neuromodulation leads used in the neuromodulation stimulation system of FIG. 1;

FIG. 3 is plan view of one embodiment of an electrical neuromodulation lead that can be used in the neuromodulation stimulation system of FIG. 1;

FIG. 3A is a cross-sectional view of the electrical neuromodulation lead of FIG. 3, taken along the line 3A-3A;

FIG. 4 is plan view of another embodiment of an electrical neuromodulation lead that can be used in the neuromodulation stimulation system of FIG. 1;

FIG. 4A is a cross-sectional view of the electrical neuromodulation lead of FIG. 4, taken along the line 4A-4A;

FIG. 5 is plan view of still another embodiment of an electrical neuromodulation lead that can be used in the neuromodulation stimulation system of FIG. 1;

FIG. 6 is plan view of an embodiment of an optical neuromodulation lead that can be used in the neuromodulation stimulation system of FIG. 1;

FIG. 6A is a magnified view of the distal end of the optical neuromodulation lead of FIG. 6, taken along the line 6A-6A;

FIG. 7 is plan view of another embodiment of an optical neuromodulation lead that can be used in the neuromodulation stimulation system of FIG. 1;

FIG. 7A is a magnified view of the distal end of the optical neuromodulation lead of FIG. 7, taken along the line 7A-7A;

FIG. 8 is plan view of still another embodiment of an optical neuromodulation lead that can be used in the neuromodulation stimulation system of FIG. 1;

FIG. 9 is plan view of yet another embodiment of an optical neuromodulation lead that can be used in the neuromodulation stimulation system of FIG. 1;

FIG. 10 is a plan view of an optical system that can be used in the optical neuromodulation lead in FIG. 9;

FIG. 11 is plan view of yet another embodiment of an optical neuromodulation lead that can be used in the neuromodulation stimulation system of FIG. 1;

FIG. 11A is a magnified view of the distal end of the optical neuromodulation lead of FIG. 11, taken along the line 11A-11A;

FIG. 12 is plan view of yet another embodiment of an optical neuromodulation lead that can be used in the neuromodulation stimulation system of FIG. 1;

FIG. 13 is plan view of an embodiment of a hybrid electrical/optical neuromodulation lead that can be used in the neuromodulation stimulation system of FIG. 1;

FIG. 14 is a block diagram of the internal components of the FIM of FIG. 1;

FIG. 15 is front view of a remote control (RC) used in the neuromodulation stimulation system of FIG. 1;

FIG. 16 is a block diagram of the internal components of the RC of FIG. 5;

FIG. 17 is a plan view of the neuromodulation system of FIG. 1 in use within the spinal column a patient for treating chronic pain;

FIG. 18 is a cross-sectional view showing the use of an optical neuromodulation lead in modulating the dorsal column (DC) nerve fibers of a spinal cord;

FIG. 19 is a cross-sectional view showing the use of an optical neuromodulation lead in modulating the dorsal root (DR) nerve fiber;

FIG. 20 is a cross-sectional view showing the use of an optical neuromodulation lead in modulating a dorsal root ganglion (DRG);

FIG. 21 is a cross-sectional view showing the use of an optical neuromodulation blanket in modulating a dorsal root ganglion (DRG);

FIG. 22 is a plan view of the neuromodulation system of FIG. 1 in use within the brain a patient for treating a variety of ailments;

FIG. 23 is a plan view showing the combined use of an electrical neuromodulation lead and an optical neuromodulation lead to modulate dorsal root (DR) nerve fibers;

FIG. 24 is a plan view showing the use of hybrid electrical/optical neuromodulation leads to modulate dorsal root (DR) nerve fibers;

FIG. 25 is a plan view showing the combined use of one electrical neuromodulation lead and two optical neuromodulation leads to modulate dorsal column (DC) nerve fibers and dorsal root (DR) nerve fibers;

FIG. 26 is a plan view showing the combined use of two electrical neuromodulation leads and one optical neuromodulation lead to modulate dorsal column (DC) nerve fibers and dorsal root (DR) nerve fibers;

FIG. 27 is a plan view showing the combined use of an electrical neuromodulation lead and an optical neuromodulation lead to modulate nerve fibers within a vagal nerve;

FIG. 28 is a diagram showing the conveyance of low-level laser energy to condition a neural axon, and the conveyance of electrical energy to stimulate the conditioned neural axon;

FIG. 29 is a diagram showing the conveyance of electrical energy to condition a plurality of neural axons, and the conveyance of low-level laser energy to stimulate one of the conditioned neural axons;

FIG. 30 is a diagram showing an arrangement involving the conveyance of electrical energy and low-level laser energy to control inherently evoked action potentials in a population of neural axons;

FIG. 31 is a diagram showing another arrangement involving the conveyance of electrical energy and low-level laser energy to control inherently evoked action potentials in a population of neural axons;

FIG. 32 is a diagram showing the use of electrical energy to bi-directionally evoke action potentials in a neural axon, while using low-level laser energy to the block the action potentials in one direction; and

FIG. 33 is a plan view showing the use of a hybrid electrical/optical neuromodulation lead to modulate nerve fibers within a vagal nerve.

### DETAILED DESCRIPTION OF THE EMBODIMENTS

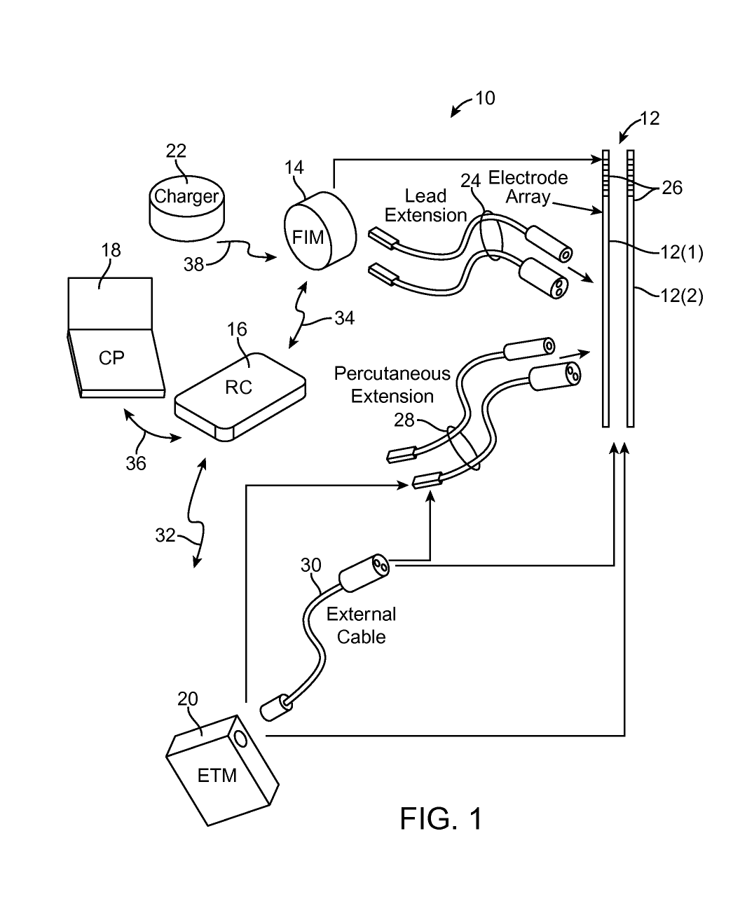

Turning first to FIG. 1, an exemplary neuromodulation system 10 is used to treat any one of a variety of ailments using low-level laser energy, and in some cases, a combination of the laser energy and electrical energy to treat any of the ailments, as well as to prevent or minimize any side-effects.

The system 10 generally includes a plurality of implantable neuromodulation leads 12, a fully implantable modulator (FIM) 14, an external control device in the form of a remote controller RC 16, a clinician's programmer (CP) 18, an external trial stimulator (ETS) 20, and an external charger 22.

The FIM 14 is physically connected via one or more lead extensions 24 to the neuromodulation leads 12, which carry a plurality of neuromodulation elements 26. Although twp neuromodulation leads 12 are illustrated, it should be appreciated that less or more neuromodulation leads 12 can be provided. As will be described in further detail below, the FIM 14 includes circuitry that delivers appropriate energy to the neuromodulation elements 26 in accordance with a set of neuromodulation parameters. The energy delivered by the FIM 14 will depend upon the nature of the neuromodulation elements 26, as will be discussed in further detail below.

The ETM 20 may also be physically connected via one or more lead extensions 28 and/or one or more external cables 30 to the neuromodulation leads 12. The ETM 20, which has similar circuitry as that of the FIM 14, also delivers appropriate energy to the neuromodulation elements 26 in accordance with a set of neuromodulation parameters. The major difference between the ETM 20 and the FIM 14 is that the ETM 20 is a non-implantable device that is used on a trial basis after the neuromodulation leads 12 have been implanted and prior to implantation of the FIM 14, to test the responsiveness of the neuromodulation that is to be provided. Thus, any functions described herein with respect to the FIM 14 can likewise be performed with respect to the ETM 20.

The RC 16 may be used to telemetrically control the ETM 20 via a bi-directional RF communications link 32. Once the FIM 14 and neuromodulation leads 12 are implanted, the RC 16 may be used to telemetrically control the FIM 14 via a bi-directional RF communications link 34. Such control allows the FIM 14 to be turned on or off and to be programmed with different neuromodulation parameter sets. The FIM 14 may also be operated to modify the programmed neuromodulation parameters to actively control the characteristics of the energy output by the FIM 14 to the neuromodulation elements 26.

The CP 18 provides clinician detailed neuromodulation parameters for programming the FIM 14 and ETM 20 in the operating room and in follow-up sessions. The CP 18 may perform this function by indirectly communicating with the FIM 14 or ETM 20, through the RC 16, via an IR communications link 36. Alternatively, the CP 18 may directly communicate with the FIM 14 or ETM 20 via an RF communications link (not shown). The clinician detailed neuromodulation parameters provided by the CP 18 are also used to program the RC 16, so that the neuromodulation parameters can be subsequently modified by operation of the RC 16 in a stand-alone mode (i.e., without the assistance of the CP 18). The external charger 22 is a portable device used to transcutaneously charge the FIM 14 via an inductive link 38. Once the FIM 14 has been programmed, and its power source has been charged by the external charger 22 or otherwise replenished, the FIM 14 may function as programmed without the RC 16 or CP 18 being present.

For purposes of brevity, the details of the CP 18, ETM 20, and external charger 22 will not be described herein. Details of exemplary embodiments of these devices are disclosed in U.S. Pat. No. 6,895,280, which is expressly incorporated herein by reference.

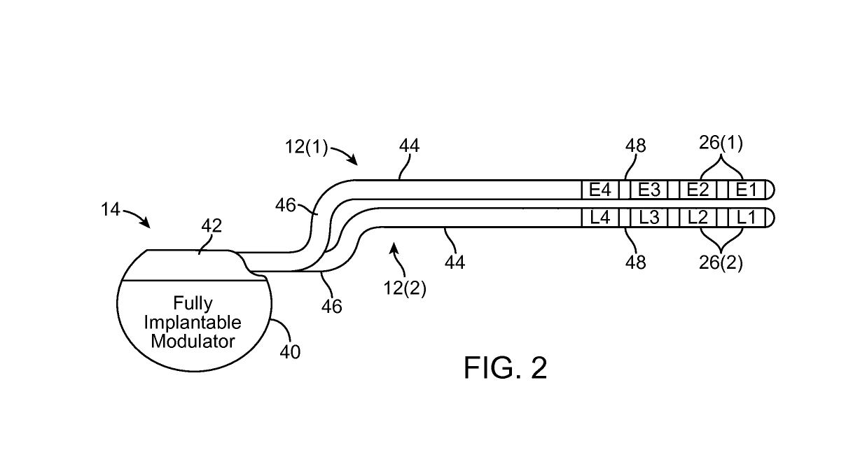

Referring now to FIG. 2, the external features of the neuromodulation leads 12 and the FIM 14 will be briefly described. The FIM 14 comprises an outer case 40 for housing the electronic and other components (described in further detail below). The outer case 40 is composed of an electrically conductive, biocompatible material, such as titanium, and forms a hermetically sealed compartment wherein the internal electronics are protected from the body tissue and fluids. In some cases, the outer case 40 may serve as an electrode. The FIM 14 further comprises a connector 42 to which the neuromodulation leads 12 mate in a manner that couples the neuromodulation elements 26 to the internal electronics (described in further detail below) within the outer case 40. To this end, the connector 42 includes two ports (not shown) for receiving the neuromodulation leads 12. In the case where the lead extensions 24 are used, the ports may instead receive the proximal ends of such lead extensions 24.

Each neuromodulation lead 12 includes an elongated lead body 44 having a proximal end 46 and a distal end 48. The lead body 44 may, e.g., have a diameter within the range of 0.03 inches to 0.07 inches and a length within the range of 10 cm to 90 cm for spinal cord stimulation applications. The lead body 44 may be composed of a suitable electrically insulative material, such as, a polymer (e.g., polyurethane or silicone), and may be extruded from as a unibody construction. Each neuromodulation lead 12 further comprises a connector 50 (not shown in FIG. 2) mounted to the proximal end 46 of the lead body 44, which mates with the connector 42 of the FIM 14 for coupling the energy generation circuitry to the neuromodulation elements 26 mounted to the distal end 48 of the lead body 44 in an in-line fashion.

The first neuromodulation lead 12(1) is designed to deliver electrical neuromodulation energy, and therefore, the neuromodulation elements 26(1) take the form of electrodes E1-E4, while the second neuromodulation lead 12(2) is designed to deliver optical neuromodulation energy, and in particular, low-level laser energy, and therefore, the neuromodulation elements 26(2) take the form of suitable optical elements L1-L4, as will be described in further detail below. In an optional embodiment described below, the functionality of the different neurostimulation leads 12 can be combined into a hybrid neuromodulation lead 12 that delivered both electrical and optical neuromodulation energy. Although each of the neuromodulation leads 12 is shown as carrying four neuromodulation elements 26, the number of neuromodulation elements 26 may be any number suitable for the application in which the neuromodulation lead 12 is intended to be used (e.g., one, two, eight, sixteen, etc.).

The FIM 14 includes circuitry that provides electrical neuromodulation energy in the form of a pulsed electrical waveform to the electrodes E1-E4 in accordance with a set of electrical neuromodulation parameters programmed into the FIM 14. Such neuromodulation parameters may comprise electrode combinations, which define the electrodes that are activated as anodes (positive), cathodes (negative), and turned off (zero), percentage of neuromodulation energy assigned to each electrode (fractionalized electrode configurations), and electrical pulse parameters, which define the pulse amplitude (measured in milliamps or volts depending on whether the FIM 14 supplies constant current or constant voltage to the electrodes E1-E4), pulse width (measured in microseconds), pulse rate (measured in pulses per second), burst rate (measured as the neuromodulation on duration X and neuromodulation off duration Y), and pulse shape.

Electrical neuromodulation will occur between two (or more) activated electrodes, one of which may be the IPG case 40. Electrical neuromodulation energy may be transmitted to the tissue in a monopolar or multipolar (e.g., bipolar, tripolar, etc.) fashion. Monopolar neuromodulation occurs when a selected one of the lead electrodes E1-E4 is activated along with the case 44 of the FIM 14, so that neuromodulation energy is transmitted between the selected electrode E1-E4 and the case 44. Bipolar neuromodulation occurs when two of the lead electrodes E1-E4 are activated as anode and cathode, so that neuromodulation energy is transmitted between the selected electrodes E1-E4. Tripolar stimulation occurs when three of the lead electrodes E1-E3 are activated, two as anodes and the remaining one as a cathode, or two as cathodes and the remaining one as an anode.

The electrical neuromodulation energy may be delivered between electrodes as monophasic electrical energy or multiphasic electrical energy. Monophasic electrical energy includes a series of pulses that are either all positive (anodic) or all negative (cathodic). Multiphasic electrical energy includes a series of pulses that alternate between positive and negative. For example, multiphasic electrical energy may include a series of biphasic pulses, with each biphasic pulse including a cathodic (negative) neuromodulation pulse and an anodic (positive) recharge pulse that is generated after the neuromodulation pulse to prevent direct current charge transfer through the tissue, thereby avoiding electrode degradation and cell trauma. That is, charge is conveyed through the electrode-tissue interface via current at an electrode during a neuromodulation period (the length of the neuromodulation pulse), and then pulled back off the electrode-tissue interface via an oppositely polarized current at the same electrode during a recharge period (the length of the recharge pulse).

The FIM 14 also includes circuitry that provides optical neuromodulation energy in the form of low-level laser energy to the optical elements L1-L4 in accordance with a set of optical neuromodulation parameters programmed into the FIM 14. The optical neuromodulation energy may be continuous or pulsed. Such neuromodulation parameters may comprise the combination of optical elements L1-L4 to be activated, optical intensity (measured in watts per centimeter squared), optical wavelength (measured in nanometers), radiation duration (measured in seconds), and burst rate (measured as the neuromodulation on duration X and neuromodulation off duration Y). In the case where laser LEDs will be used, the FIM 14 may alternatively have circuit that provides electrical energy to the laser LEDs in accordance with electrical parameters transformable to the desired optical neuromodulation parameters.

The neuromodulation effect that the application of the electrical or optical neuromodulation energy to neural tissue will depend mainly on the frequency or wavelength of the neuromodulation energy. For example, the application of low frequency pulsed electrical energy (1 Hz-500 Hz) is known to increase the excitability of neural tissue, whereas the application of high frequency pulsed electrical energy (1 KHz-50 KHz, preferably in the range of 3 KHz-15 KHz) is known to decrease the excitability of neural tissue. The application of continuous high frequency electrical energy (e.g., sinuosoidal) is also known to decrease the excitability of neural tissue. The application of optical energy (whether continuous or pulsed) having a short wavelength (600 nm-1200 nm, and preferably 600 nm-1000 nm) is known to decrease the excitability of neural tissue, whereas the application of optical energy (whether continuous or pulsed) have a long wavelength (1500 nm-2500 nm) is known to increase the excitability of neural tissue.

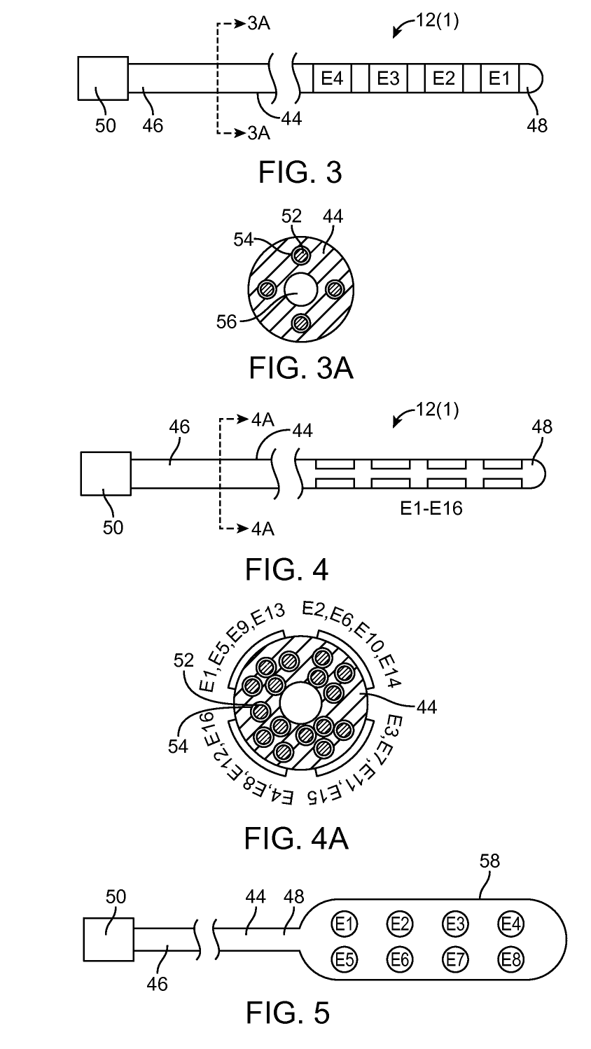

Referring to FIGS. 3-5, different types of neurostimulation leads 12(1) for delivering electrical neuromodulation energy will now be described.

In the embodiment illustrated in FIG. 3, the neurostimulation lead 12(1) is a percutaneous lead, and each of the electrodes E1-E4 takes the form of a cylindrical ring element composed of an electrically conductive, non-corrosive, material, such as, e.g., platinum, platinum iridium, titanium, or stainless steel, which is circumferentially disposed about the lead body 44. In this manner, the electrodes E1-E4 are radially non-directional in that the electrical energy is conveyed radially uniformly around the electrode E1-E4.

As shown in FIG. 3A, the neuromodulation lead 12(1) includes a plurality of electrical conductors 52 extending through individual lumens 54 within the lead body 44 and connected between the connector 50 and the electrodes E1-E4 using suitable means, such as welding, thereby electrically coupling the proximally-located connector 50 with the distally-located electrodes E1-E4. The neuromodulation lead 12(1) further includes a central lumen 56 that may be used to accept an insertion stylet (not shown) to facilitate lead implantation. The connector 50 includes electrical terminals (not shown) hardwired to the respective conductors 52 and capable of mating with corresponding electrical terminals (not shown) on the connector 42 of the FIM 14 (shown in FIG. 2). Thus, electrical energy can be conveyed from the IMN 14 to the connector 50, along the conductors 52 to the electrodes E1-E4. Alternatively, rather than having a single connector 50, a plurality of terminals (not shown) may be mounted to the proximal end 46 of the lead body 44 in respective electrical communication to the electrodes E1-E4 via the conductors 52.

In the embodiment illustrated in FIG. 4, the electrodes are segmented, as described in U.S. patent application Ser. No. 13/212,063, entitled “User Interface for Segmented Neurostimulation Leads,” which is expressly incorporated herein by reference. In particular, the electrodes take the form of segmented electrodes that are circumferentially and axially disposed about the lead body 44. For example, the neuromodulation lead 12(1) may carry sixteen electrodes, arranged as four rings of electrodes (the first ring consisting of electrodes E1-E4; the second ring consisting of electrodes E5-E8; the third ring consisting of electrodes E9-E12; and the fourth ring consisting of E13-E16) or four axial columns of electrodes (the first column consisting of electrodes E1, E5, E9, and E13; the second column consisting of electrodes E2, E6, E10, and E14; the third column consisting of electrodes E3, E7, E11, and E15; and the fourth column consisting of electrodes E4, E8, E12, and E16). In this manner, the electrodes E1-E16 are radially directional, such that the electrical energy may be selectively conveyed both linearly and circumferentially along the lead body 44. As shown in FIG. 4A, the neuromodulation lead 12(1) includes a plurality of electrical conductors 52 extending through individual lumens 54 within the lead body 44 and connected between the connector 50 and the electrodes E1-E16 using suitable means, such as welding, thereby electrically coupling the proximally-located connector 50 with the distally-located electrodes E1-E16.

In the embodiment illustrated in FIG. 5, the neurostimulation lead 12(1) is a surgical paddle lead that includes a distally-located paddle 58 on which disk-shaped electrodes E1-E8 are arranged in a two-dimensional array in two columns. In this manner, the electrodes E1-E8 are directional, such that the electrical energy may be focused on one lateral side of the paddle 58. The connector 50 and the electrodes E1-E8 may be coupled to each other via electrical conductors 52, as described above with respect to FIG. 3A.

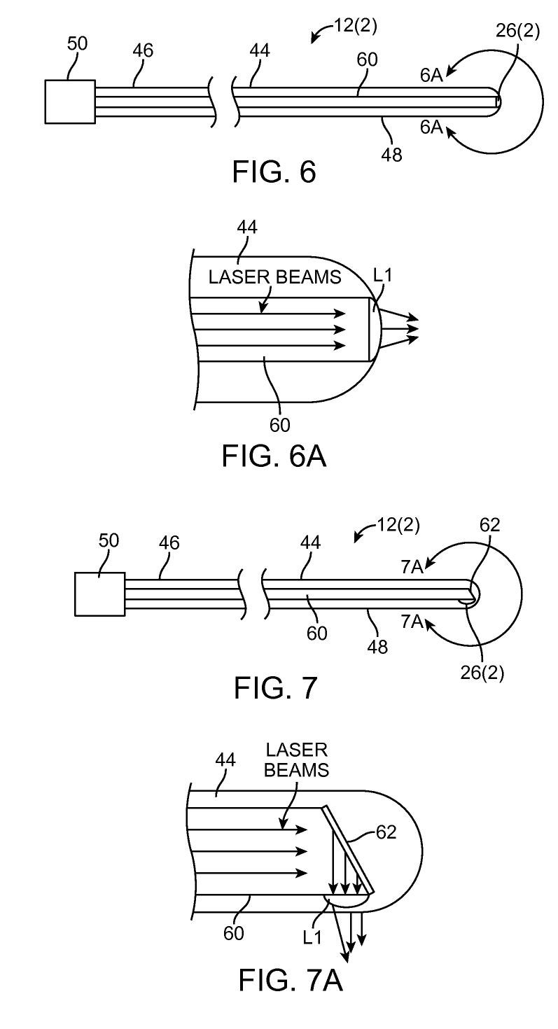

Referring to FIGS. 6-12, different types of neurostimulation leads 12(2) for delivering optical neuromodulation energy will now be described.

In the embodiment illustrated in FIG. 6, the neurostimulation lead 12(2) is a percutaneous lead, and each of the optical elements L1-L4 takes the form of a focusing element, such as a lens, that focuses the laser energy on the target tissue. The neuromodulation lead 12(2) includes an optical fiber 60 extending through the lead body 44 and connected between the connector 50 and the lens L1 using suitable means, thereby optically coupling the proximally-located connector 50 with the distally-located lens L1. Thus, optical energy can be conveyed from the IMN 14 to the connector 50, along the optical fiber 56, and then through the lens L1. The neuromodulation lead 12(2) may further include a central lumen (not shown) that may be used to accept an insertion stylet (not shown) to facilitate lead implantation in the same manner described above with respect to FIG. 3. The connector 50 takes the form of an optical fiber connector that includes an optical coupler (not shown) connected to the optical fiber 60 and capable of mating with a corresponding optical coupler (not shown) on the connector 42 of the FIM 14. In the embodiment illustrated in FIG. 6, the lens L1 is disposed at the distal tip of the lead body 44 and focuses the laser energy along the longitudinal axis of the lead body 44.

In an alternative embodiment illustrated in FIG. 7, the neuromodulation lead 12(2) further includes a reflecting element (e.g., mirror) 62 for directing the laser energy from the optical fiber 56 to the lens L1 in a lateral direction relative to the longitudinal axis of the lead body 44.

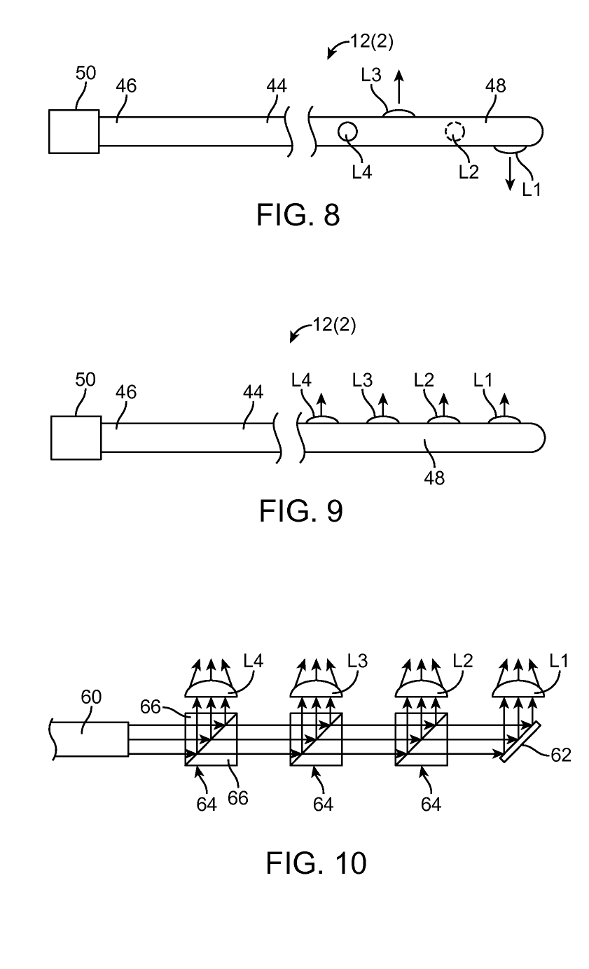

Although the lens L1 is shown in FIGS. 6 and 7 to be disposed at the distal tip of the lead body 44, it should be noted that one or more lenses L1 can be disposed laterally along the distal end 48 of the lead body 44. For example, as illustrated in FIG. 8, multiple lenses L1-L4 can be disposed along the distal end 48 of the lead body 44. In this embodiment, the neuromodulation lead 12(2) includes multiple optical fibers (not shown) extending through the lead body 44 in a parallel manner and connected between the connector 50 and the respective lenses L1-L4 using suitable means. In this case, the connector 50 includes multiple optical couplers (not shown) connected to respective optical fiber and capable of mating with corresponding optical couplers (not shown) on the connector 42 of the FIM 14. The laser energy is conveyed lateral to the longitudinal axis of the lead body 44, and thus, reflectors (not shown) are used to laterally direct the laser energy from the optical fibers 56 to the respective lenses L1-L4. Thus, optical energy can be conveyed from the IMN 14 to the connector 50, along the optical fibers, and then reflected from the reflectors through the lenses L1-L4. In the illustrated embodiment, the four lenses L1-L4 are radially oriented from each other by 90 degrees, such that laser energy can be selectively conveyed in one of four radial directions, depending on the particular lens from which the laser energy is conveyed. Alternatively, the lenses L1-L4 may be disposed on only one lateral side of the lead body 44, such that they all convey laser energy in the same radial direction, as illustrated in FIG. 9.

In an alternative embodiment, rather than a plurality of parallel optical fibers, a single optical fiber is used to connect the connector 50 and the respective lenses L1-L4. In this case, as shown in FIG. 10, the neuromodulation lead 12(2) further includes a plurality of beam splitters 64 (in this case, three) in series with the optical fiber 60 for coupling optical energy carried by the optical fiber 60 to the lenses L1-L4. The beam splitter 64 can be formed, e.g., by bonding two triangular glass prisms 66 together. Alternatively, the beam splitter 64 may be formed of a partially silvered mirror or a dichroic mirrored prism. In any event, a portion of the optical energy incident on the beam splitter 64 will be reflected in a direction towards the respective lens, while the remaining portion is transmitted along the longitudinal axis of the lead body 44 to the next beam splitter 64. A reflector 62 is used to reflect the remaining optical energy to the distal-most lens L1.

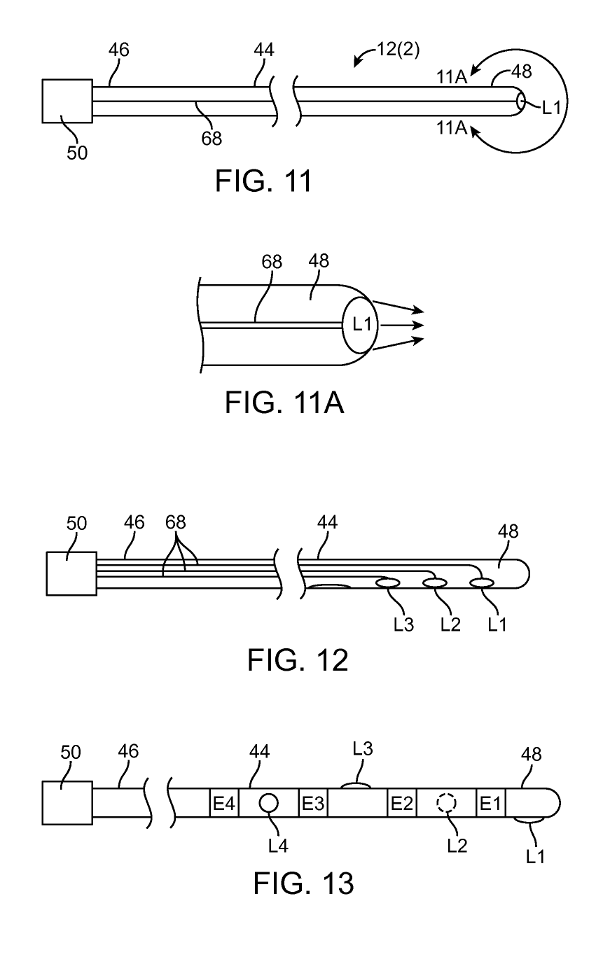

Although the optical elements L1-L4 has been described as taking the form of a lens that focuses the laser energy on the target tissue, the optical elements L1-L4 can take other forms, such as a laser light emitting diode (LED), which operates as both a generator of the laser energy and a lens. In particular, as illustrated in FIG. 11, a laser LED L1 is mounted to the distal tip of the lead body 44. In this case, the neuromodulation lead 12(2) includes an electrical conductor 68, similar to the conductor 52 discussed above with respect to neuromodulation element 26(1), extending through the lead body 44 between the connector 50 and the laser LED L1. Thus, electrical energy can be conveyed from the IMN 14 to the connector 50, along the conductor 52 and to the laser LED L1, where it is transformed into laser energy. As illustrated in FIG. 12, three laser LEDs L1-L3 are disposed along the distal end 48 of the lead body 44. In this case, the neuromodulation lead 12(2) includes there electrical conductors 68 extending through the lead body 44 between the connector 50 and the respective laser LEDs L1-L3. Thus, electrical energy can be conveyed from the IMN 14 to the connector 50, along the conductors 52 and to the laser LEDs L1-L3, where it is transformed into laser energy.

Although the neuromodulation leads 12 have been described as being capable of delivering either electrical neuromodulation energy or delivering optical neuromodulation energy, one or more of the neuromodulation leads 12 can be capable of selectively delivering both electrical modulation energy and optical neuromodulation energy. For example, referring to FIG. 13, a neuromodulation lead 12(3) comprises a plurality of electrical neuromodulation elements 26(1) in the form of electrodes E1-E4 and a plurality of optical neuromodulation elements 26(2) in the form of lenses (or laser LEDs) L1-L4 extending along the distal end 48 of the lead body 44. In the preferred embodiment, the electrodes E1-E4 and lenses L1-L4 are interleaved with each other so that they occupy virtually the same space. Alternatively, the electrodes E1-E4 can be grouped together, and the lenses L1-L4 can be grouped together, such that all of the electrodes E1-E4 are proximal to all the lenses L1-L4, or vice versa. The neuromodulation lead 12(3) includes electrical conductors (not shown) and optical fiber(s) (not shown) (or electrical conductors in the case where laser LEDs are used) extending through the lead body 44 between the connector 50 and the respective electrodes E1-E4 and lenses L1-L4 in the same manner described above with respect to the neuromodulation leads 12(1) and 12(2). The neuromodulation lead 12(2) may also include reflectors 62 (shown in FIG. 7) to laterally direct the optical energy from the optical fiber(s) to the lenses L1-L4, and if only one optical fiber 56 is utilized, beam splitters 64 (shown in FIG. 10) to distribute the optical energy from the optical fiber to the lenses L1-L4.

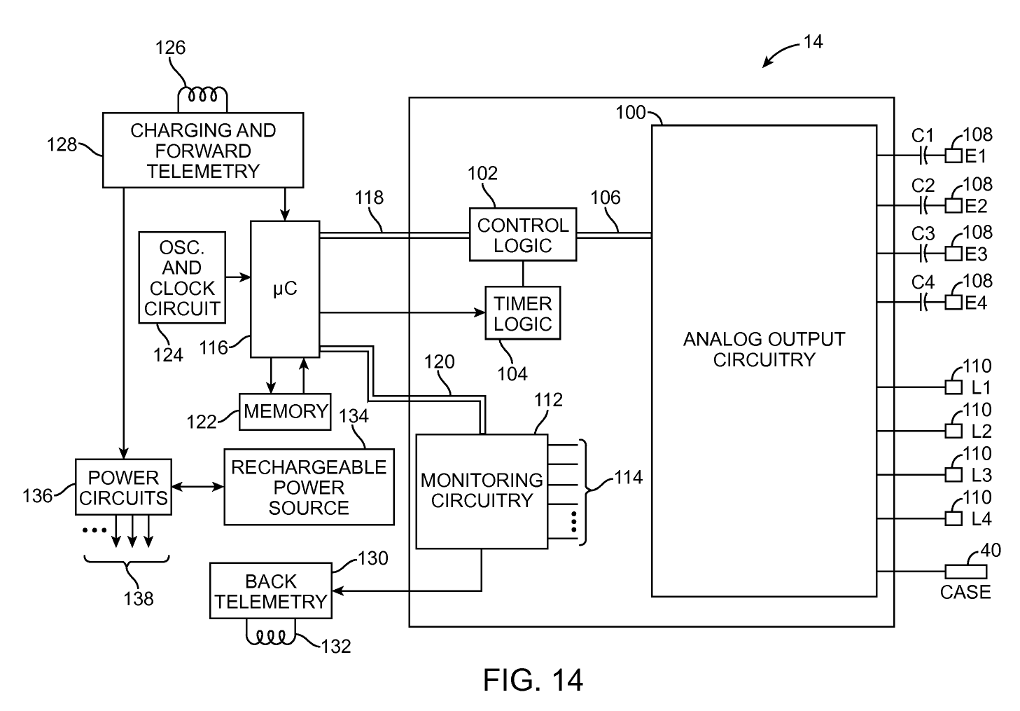

Turning next to FIG. 14, the main internal components of the FIM 14 will now be described. The FIM 14 comprises analog output circuitry 100 configured for selectively generating electrical neuromodulation energy in accordance with a defined pulsed waveform having a specified pulse amplitude, pulse rate, pulse width, pulse shape, and burst rate, and optical neuromodulation energy in accordance with an optical intensity, optical wavelength, radiation duration, and burst rate (or alternatively, equivalent electrical parameters if laser LEDs are used) under control of control logic 102 and timer logic 104 over data bus 106. The electrical neuromodulation energy generated by the analog output circuitry 100 is output via capacitors C1-C4 to electrical terminals 108 corresponding to the electrodes E1-E4, while the optical neuromodulation energy generated by the stimulation output circuitry 100 is output to optical terminals 110 corresponding to optical elements L1-L4. Of course, the number of electrical terminals 108 and optical terminals 110 will depend on the number of electrodes and lenses to which they will be coupled.

With respect to generating pulsed electrical neuromodulation energy, the analog output circuitry 50 may either comprise independently controlled current sources for providing electrical pulses of a specified and known amperage to or from the electrodes E1-E4, or independently controlled voltage sources for providing electrical pulses of a specified and known voltage at the electrodes E1-E4. The operation of this analog output circuitry, including alternative embodiments of suitable output circuitry for performing the same function of generating electrical pulses of a prescribed amplitude and width, is described more fully in U.S. Pat. Nos. 6,516,227 and 6,993,384, which are expressly incorporated herein by reference. The analog output circuitry 50 may optionally generate high frequency blocking electrical energy as described in U.S. patent application Ser. No. 12/819,107, entitled “Spatially Selective Nerve Stimulation in High-Frequency Nerve Conduction Block and Recruitment,” and U.S. Provisional Patent Application Ser. No. 61/646,773, entitled “System and Method for Shaped Phased Current Delivery,” which are expressly incorporated herein by reference. With respect to generating optical neuromodulation energy, the analog output circuitry 50 may comprise any conventional miniaturized laser generation device.

The FIM 14 also comprises monitoring circuitry 112 for monitoring the status of various nodes or other points 114 throughout the FIM 14, e.g., power supply voltages, temperature, battery voltage, and the like. The FIM 14 further comprises processing circuitry in the form of a microcontroller (μC) 116 that controls the control logic 102 over data bus 118, and obtains status data from the monitoring circuitry 112 via data bus 120. The microcontroller 116 additionally controls the timer logic 106. The FIM 14 further comprises memory 122 and oscillator and clock circuit 124 coupled to the microcontroller 116. The microcontroller 116, in combination with the memory 122 and oscillator and clock circuit 124, thus comprise a microprocessor system that carries out a program function in accordance with a suitable program stored in the memory 122. Alternatively, for some applications, the function provided by the microprocessor system may be carried out by a suitable state machine.

Thus, the microcontroller 116 generates the necessary control and status signals, which allow the microcontroller 116 to control the operation of the FIM 14 in accordance with a selected operating program and neuromodulation parameters. In controlling the operation of the FIM 14, the microcontroller 116 is able to individually generate neuromodulation energy at the electrodes E1-E4 and optical elements L1-L4 using the analog output circuitry 100, in combination with the control logic 102 and timer logic 106, to control neuromodulation parameters of the generated neuromodulation energy.

The FIM 14 further comprises an alternating current (AC) receiving coil 126 for receiving programming data (e.g., the operating program and/or neuromodulation parameters) from the RC 16 and/or CP 18 in an appropriate modulated carrier signal, and charging and forward telemetry circuitry 128 for demodulating the carrier signal it receives through the AC receiving coil 126 to recover the programming data, which programming data is then stored within the memory 122, or within other memory elements (not shown) distributed throughout the FIM 14.

The FIM 14 further comprises back telemetry circuitry 130 and an alternating current (AC) transmission coil 132 for sending informational data sensed through the monitoring circuitry 112 to the RC 16 and/or CP 18. The back telemetry features of the FIM 14 also allow its status to be checked. For example, when the RC 16 and/or CP 18 initiates a programming session with the FIM 14, the capacity of the battery is telemetered, so that the RC 16 and/or CP 18 can calculate the estimated time to recharge. Any changes made to the current stimulus parameters are confirmed through back telemetry, thereby assuring that such changes have been correctly received and implemented within the implant system. Moreover, upon interrogation by the RC 16 and/or CP 18, all programmable settings stored within the FIM 14 may be uploaded to the RC 16 and/or CP 18.

The FIM 14 further comprises a rechargeable power source 134 and power circuits 136 for providing the operating power to the FIM 14. The rechargeable power source 134 may, e.g., comprise a lithium-ion or lithium-ion polymer battery. The rechargeable battery 134 provides an unregulated voltage to the power circuits 136. The power circuits 136, in turn, generate the various voltages 138, some of which are regulated and some of which are not, as needed by the various circuits located within the FIM 14. The rechargeable power source 134 is recharged using rectified AC power (or DC power converted from AC power through other means, e.g., efficient AC-to-DC converter circuits, also known as “inverter circuits”) received by the AC receiving coil 126. To recharge the power source 134, an external charger (not shown), which generates the AC magnetic field, is placed against, or otherwise adjacent, to the patient's skin over the implanted FIM 14. The AC magnetic field emitted by the external charger induces AC currents in the AC receiving coil 126. The charging and forward telemetry circuitry 128 rectifies the AC current to produce DC current, which is used to charge the power source 134. While the AC receiving coil 126 is described as being used for both wirelessly receiving communications (e.g., programming and control data) and charging energy from the external device, it should be appreciated that the AC receiving coil 126 can be arranged as a dedicated charging coil, while another coil, such as coil 132, can be used for bi-directional telemetry.

It should be noted that rather than being fully contained and powered, the FIM may be an implantable receiver-stimulator (not shown) connected to neuromodulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the neuromodulation energy in accordance with the control signals.

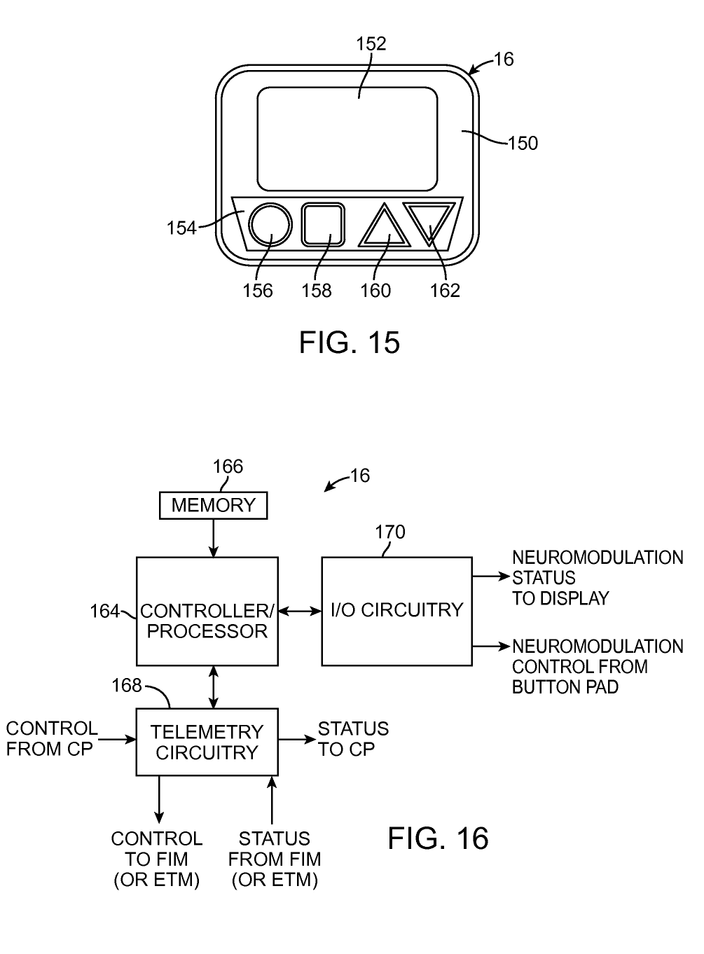

Referring now to FIG. 15, one exemplary embodiment of an RC 16 will now be described. As previously discussed, the RC 16 is capable of communicating with the FIM 14, CP 18, or ETM 20. The RC 16 comprises a casing 150, which houses internal componentry (including a printed circuit board (PCB)), and a lighted display screen 152 and button pad 154 carried by the exterior of the casing 150. In the illustrated embodiment, the display screen 152 is a lighted flat panel display screen, and the button pad 154 comprises a membrane switch with metal domes positioned over a flex circuit, and a keypad connector connected directly to a PCB. In an optional embodiment, the display screen 152 has touch screen capabilities. The button pad 154 includes a multitude of buttons 156, 158, 160, and 162, which allow the FIM 14 to be turned ON and OFF, provide for the adjustment or setting of neuromodulation parameters within the FIM 14, and provide for selection between screens.

In the illustrated embodiment, the button 106 serves as an ON/OFF button that can be actuated to turn the FIM 14 ON and OFF. The button 158 serves as a select button that allows the RC 16 to switch between screen displays and/or parameters. The buttons 160 and 162 serve as up/down buttons that can be actuated to increment or decrement any of neuromodulation parameters of the pulse generated by the FIM 14, including pulse amplitude, pulse width, and pulse rate. For example, the selection button 158 can be actuated to place the RC 16 in an “Electrical Modulation Adjustment Mode,” during which any of the electrical neuromodulation parameters can be selected and adjusted via the up/down buttons 160, 162, or an “Optical Modulation Adjustment Mode,” during which any of the electrical neuromodulation parameters can be selected and adjusted via the up/down buttons 160, 162. Alternatively, dedicated up/down buttons can be provided for each neuromodulation parameter. Rather than using up/down buttons, any other type of actuator, such as a dial, slider bar, or keypad, can be used to increment or decrement the neuromodulation parameters. Further details of the functionality and internal componentry of the RC 16 are disclosed in U.S. Pat. No. 6,895,280, which has previously been incorporated herein by reference.

Referring to FIG. 16, the internal components of an exemplary RC 16 will now be described. The RC 16 generally includes a controller/processor 164 (e.g., a microcontroller), memory 166 that stores an operating program for execution by the controller/processor 164, as well as neuromodulation parameter sets in a navigation table (described below), input/output circuitry, and in particular, telemetry circuitry 168 for outputting neuromodulation parameters to the FIM 14 and receiving status information from the FIM 14, and input/output circuitry 170 for receiving stimulation control signals from the button pad 154 and transmitting status information to the display screen 152 (shown in FIG. 15). As well as controlling other functions of the RC 16, which will not be described herein for purposes of brevity, the processor 114 generates new neuromodulation parameter sets in response to the user operation of the button pad 154. These new neuromodulation parameter sets would then be transmitted to the FIM 14 via the telemetry circuitry 118. Notably, while the controller/processor 64 is shown in FIG. 15 as a single device, the processing functions and controlling functions can be performed by a separate controller and processor. Further details of the functionality and internal componentry of the RC 16 are disclosed in U.S. Pat. No. 6,895,280, which has previously been incorporated herein by reference.

Having described the structure and function of the neuromodulation system 10, various techniques for using the neuromodulation system 10 to treat patients having in any of a variety of ailments will now be described. In these methods, low-level laser energy having a wavelength in the range of 600 nm-2500 nm is conveyed from the optical neuromodulation lead 12(2) or the hybrid neuromodulation lead 12(3) to a neuronal element to treat the ailment. The neuronal element may be a neural structure found in the central nervous system (CNS), such as the brain, spinal cord, or a region thereof, or neural structure found in the peripheral nervous system (PNS), such as a spinal nerve or a dorsal root ganglion (DRG). Or the neuronal element may be a part of a neuron, such as an axon or cell body. Because low-level laser energy can be better focused on target neural structures than electrical energy, the use of laser energy may reduce side-effects that may otherwise be caused by inadvertently modulating non-target neural structures.

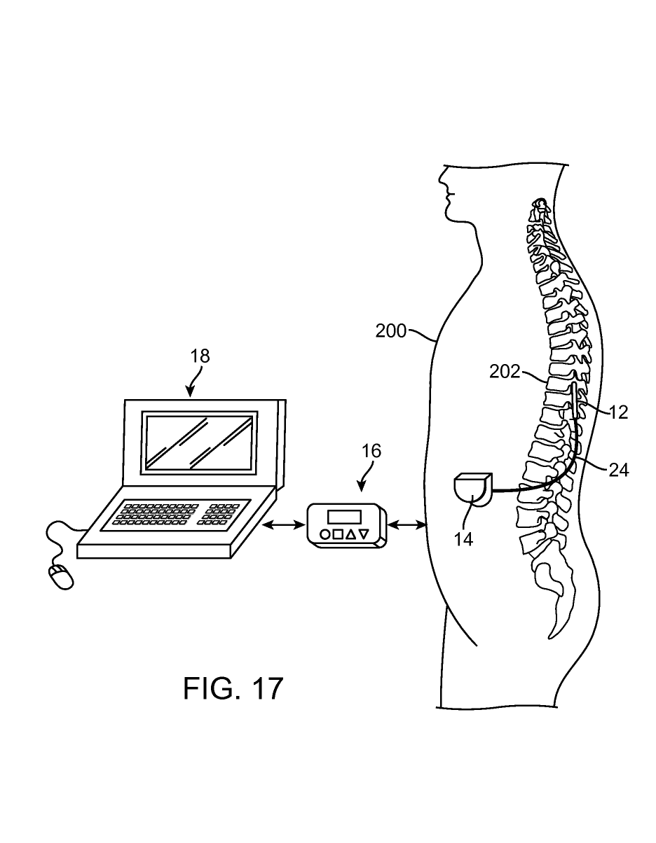

In one method for treating chronic pain, the spinal cord and/or surrounding neural structures may be modulated by implanting the optical neuromodulation lead 12(2) within the spinal column 202 of a patient 200, as shown in FIG. 17. The preferred placement of the neuromodulation lead 12(2) is in the epidural space 204 (not shown in FIG. 17) of the patient 200. The percutaneous neuromodulation lead 12(2) can be introduced, with the aid of fluoroscopy, into the epidural space through a Touhy-like needle, which passes through the skin, between the desired vertebrae, and into the epidural space above the dura layer. In many cases, a stylet, such as a metallic wire, is inserted into a lumen running through the center of the neuromodulation lead 12(2) to aid in insertion of the lead through the needle and into the epidural space 204. The stylet gives the lead rigidity during positioning, and once the neuromodulation lead 12(2) is positioned, the stylet can be removed after which the lead becomes flaccid. In the case where a surgical paddle lead is alternatively used in place of the percutaneous lead, it can be implanted within the spinal column 202 using a surgical procedure, and specifically, a laminectomy, which involves removal of the laminar vertebral tissue to allow both access to the dura layer and positioning of the neuromodulation lead 12(2).

After proper placement of the neuromodulation lead 12(2) at the target area of the spinal column 202, the neuromodulation lead 12(2) is anchored in place to prevent movement of the neuromodulation lead 12. To facilitate the location of the FIM 14 away from the exit point of the neuromodulation lead 12 implanted within the spinal column 202, the lead extension 24 may be used. Whether lead extensions are used or not, the proximal ends of the neuromodulation lead 12 exiting the spinal column 202 is passed through one or more tunnels (not shown) subcutaneously formed along the torso of the patient 200 to a subcutaneous pocket (typically made in the patient's abdominal or buttock area) where the FIM 14 is implanted. The FIM 14 may, of course, also be implanted in other locations of the patient's body. A subcutaneous tunnel can be formed using a tunneling tool over which a tunneling straw may be threaded. The tunneling tool can be removed, the neuromodulation lead 12 threaded through the tunneling straw, and then the tunneling straw removed from the tunnel while maintaining the neuromodulation lead 12 in place within the tunnel.

The neuromodulation lead 12 is then connected directly to the FIM 14 by inserting the connector 50 of the neuromodulation lead 12 within the connector port located on the connector 42 of the FIM 14 or connected to lead extension 24, which is then inserted into the connector port of the FIM 14. The FIM 14 can then be operated to generate the laser energy, which is delivered, through the optical elements L1-L4, to the targeted tissue. As there shown, the CP 18 communicates with the FIM 14 via the RC 16, thereby providing a means to control and reprogram the FIM 14.

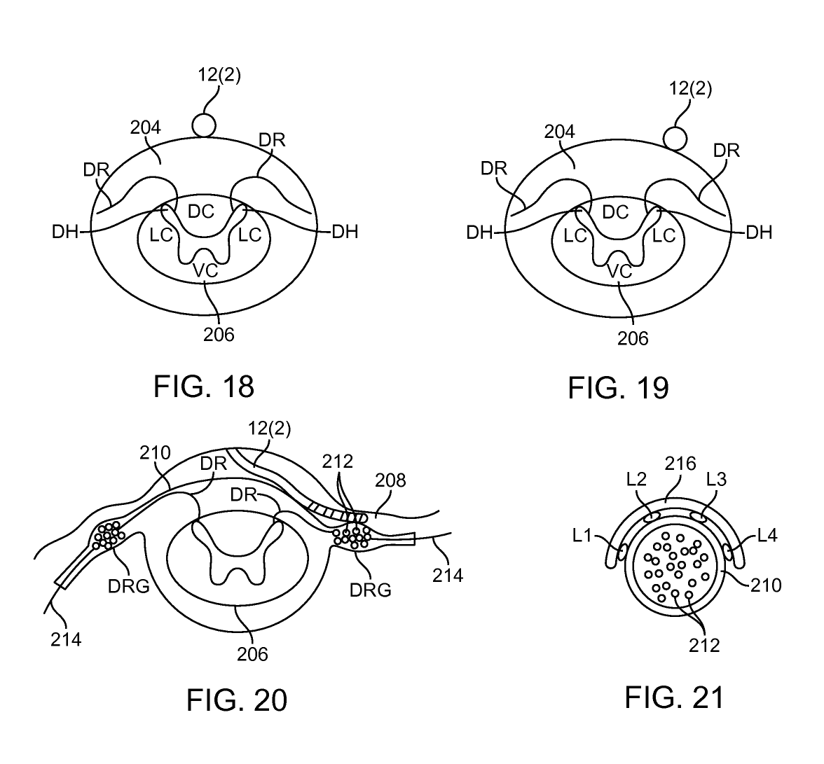

In the embodiment illustrated in FIG. 18, the optical neuromodulation lead 12(2) is located in the epidural space 204 above the dorsal columns (DC) of the spinal cord 206. In this manner, laser energy may be conveyed from the neuromodulation lead 12(2) to modulate the dorsal column (DC) nerve fibers. As shown in FIG. 19, the optical neuromodulation lead 12(2) may be located in the epidural space 204 above one of the dorsal root (DR) nerve fibers. In this manner, the laser energy may be conveyed from the neuromodulation lead 12(2) to modulate the DR nerve fiber. The laser energy may have a relatively low wavelength (e.g., 600 nm-1200 nm) to decrease the excitability of the DC or DR nerve fibers or a relatively high wavelength (e.g., 1500 nm-2500 nm) to increase the excitability of the DC or DR nerve fibers, thereby treating the chronic pain of the DC or DR nerve fibers that innervate the body region of the patient afflicted with the pain. Other neural structures that can be optically modulated from the epidural space 204, such as the ventral column (VC) fibers, lateral column (LC) fibers, or the dorsal horn (DH), may be modulated by placing the neuromodulation lead 12(2) within the epidural space 204 adjacent these neural structures and conveying the low-level laser energy from the properly located neuromodulation lead 12(2).

Referring to FIG. 20, the optical neuromodulation lead 12(2) may be located in the foramen 208 that extends from epidural space 204 over the dura 210 covering the DRG. Notably, the DRG is a unique neural structure in the body in that it contains the cell bodies 212 for the most somatic sensory neurons 214. This positioning of the cell body (or soma) 212 somewhat midway along the length of sensory neurons 214, and thus, may be called “pseudounipolar.” Traditionally, a cell soma provides metabolic support, but DRG soma are known to undergo subthreshold depolarization when neighbor soma 212 are invaded with afferent spikes. This means that some degree of cross-talk between the cell bodies 212 can occur in the DRG. In healthy DRG, these interactions tend to be causal, in that regular afferent activity will generate subthreshold oscillations and some spiking while the afferent signaling is present, but rarely when sensory neurons are quiet. In pathological states, such as those following nerve injury or trauma, it is believed that the DRG soma become hyperactive, such that they generate enhanced periodic subthreshold membrane oscillations, often independent of afferent activity. In the hyperactive state, the soma have increased metabolic needs, and these needs may lead to oxygen debt and reduced mitochrondrial performance with the sensory neurons. This, in turn, can lead to ectopic electrical spiking within the sensory neurons. The action potentials resulting from the ectopic electrical spiking then feed into the dorsal horn laminae and are believed to hypersensitize these neural structures. This hypersensitization may then lead to chronic pain.

It is believed that the application of low-level laser energy to the hyperactive DRG would restore normal function to the sensory neurons 214, ostensibly reducing chronic pain signaling and therefore the perception and burden of chronic pain. Preferably, the wavelength of the laser energy delivered from the neuromodulation lead 12(2) is in the range of 600 nm-1200 nm, and preferably in the range of 600-900 nm, in order to decrease the excitability of the DRG. In an optional arrangement, the laser LEDs L1-L4 may be mounted on a thin flexible sheet 216 that is wrapped around the DRG, such that the laser LEDs L1-L4 are positioned directionally to face the DRG, thereby directing the low-level laser energy towards the DRG, as shown in FIG. 21.

Other neural structures of the PNS can be modulated using the neuromodulation lead 12(2). For example, the neuromodulation lead 12(2) may be implanted within the subcutaneous tissues of the lower back, directly in the region of maximum pain; e.g., placed laterally (horizontally) across the back of the patient at the L4-L5 vertebral levels overlying the paraspinous muscles. As another example, the neuromodulation lead 12(2) may be implanted in other regions of the patient where peripheral nerves can be modulated, including the head (e.g., ONS) and cervical regions, abdomen, and limbs.

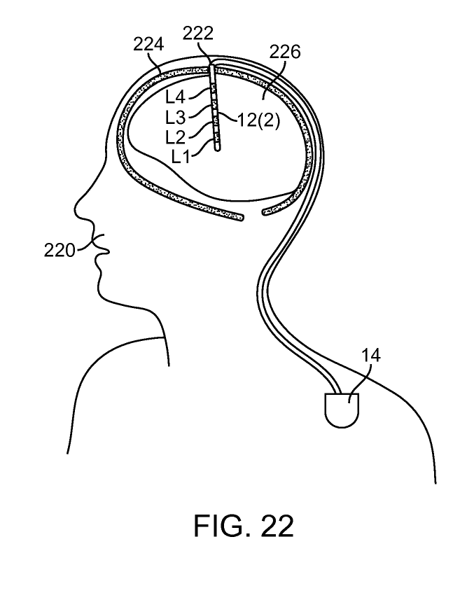

Referring to FIG. 22, in one method for treating a variety of ailments, the optical neuromodulation lead 12(2) may be advanced through a burr hole 222 formed in the cranium 224 of a patient 220, and introduced into the parenchyma of the brain 226 in a conventional manner, such that the optical elements L1-L4 are adjacent a target tissue region, the neuromodulation of which will treat the dysfunction. Due to the lack of space near the location where the neuromodulation lead 12(2) exits the burr hole 222, the FIM 14 is generally implanted in a surgically-made pocket either in the chest, or in the abdomen. The FIM 14 may, of course, also be implanted in other locations of the patient's body. The lead extension 24 facilitates locating the FIM 14 away from the exit point of the neuromodulation lead 12(2).

The brain structure to be modulated will depend on the ailment to be treated. For the treatment of Parkinson's disease, the thalamus, subthalamic nucleus (STN), and or globus pallidus may be modulated. For the treatment of depression, the subgenual cingulate cortex, rostral cingulate cortex, ventral striatum, nucleus accumbens, inferior thalamic peduncle, and/or lateral habernula may be modulated. For the treatment of addiction, the nucleus accumbens may be modulated. For the treatment of obesity, the ventromedial hypothalamus may be modulated. For the treatment of tremor, the ventrolateral thalamus and/or zona incerta may be modulated. For the treatment of dystonia, the posteroventral globus pallidus pars ilnternus, STN, and/or ventrolateral thalamus may be modulated. For the treatment of pain, the periventricular gray (PVG), periaqueductal gray (PAG), and/or ventrocaudal thalamus may be modulated. For the treatment of epilepsy, the cerebellum, centromedian nucleus of thalamus, anterior nucleus of thalamus, STN, caudate nucleus, and/or mesial temporal lobe may be modulated. For the treatment of obsessive compulsive disorder (OCD), the ventral capsule (VC), ventral striatum (VS), and/or nucleus accumbens may be modulated. For the treatment of Tourette's syndrome, the centromedian nucleus of thalamus, pars interna of the globus internal, and/or anterior limb of internal capsule may be modulated.

In other methods, both electrical energy is conveyed from the neuromodulation lead 12(1) (or optionally, the hybrid neuromodulation lead 12(3)) to a neuronal element, and low-level laser energy having a wavelength in the range of 600 nm-2500 nm is conveyed from the optical neuromodulation lead 12(2) (or optionally, the hybrid neuromodulation lead 12(3)) to the same neuronal element. At least one of the conveyance of the electrical energy and the conveyance of the low-level laser energy treats the ailment. In this manner, the advantages of electrical neuromodulation and optical neuromodulation may be combined to more effectively modulate neuronal elements. For example, combining electrical stimulation with adjunct low level laser energy neuromodulation may enhance the performance of conventional neurostimulation for pain management.

In one method, electrical energy is conveyed to a second neuronal element, thereby modulating the second neuronal element to treat the ailment. In this case, the modulation of the first neuronal element by the low-level laser energy decreases a side-effect otherwise caused by the modulation of the first neuronal element by the electrical energy.

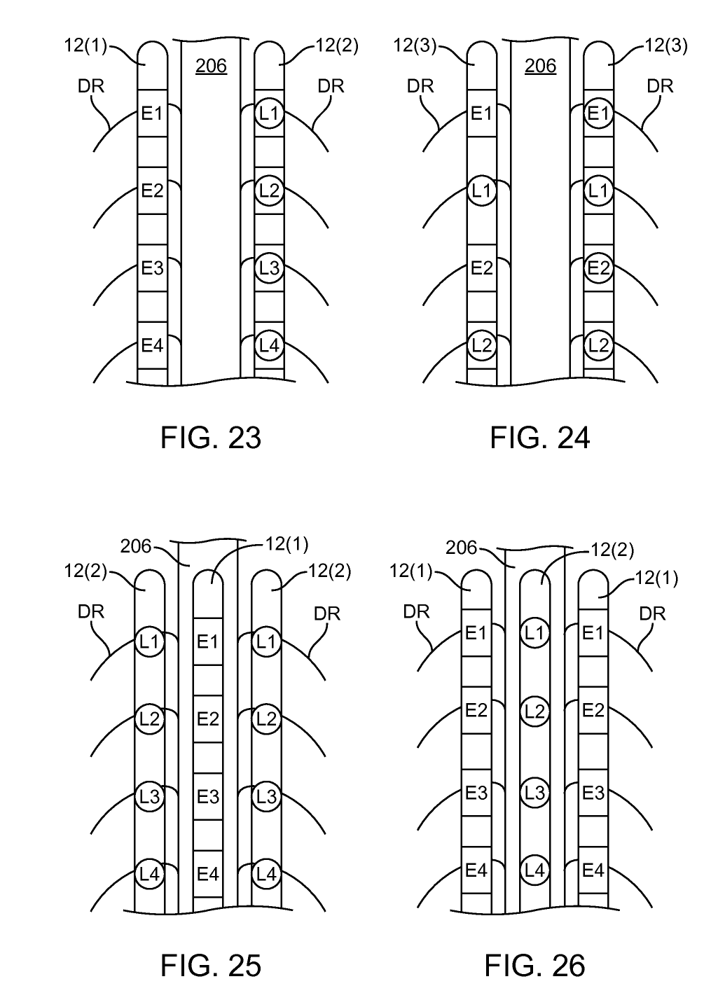

For example, as illustrated in FIG. 23, the electrical neuromodulation lead 12(1) and optical neuromodulation lead 12(2) may be epidurally located on both sides of the spinal cord 206 above the DR nerve fibers at the same vertebral level. Electrical energy having a relatively low frequency (e.g., 1 Hz-500 Hz) can be conveyed from the neuromodulation lead 12(1), thereby increasing the excitability and stimulating the left DR nerve fiber, and treating chronic pain in an anatomical region innervated by the left DR nerve fiber. However, because electrical energy is relatively unfocused, the right DR nerve fiber may be inadvertently stimulated, thereby causing a side-effect (e.g., motor movement or painful sensations). Thus, laser energy having a relatively low wavelength (e.g., 600 nm-1200 nm) can be conveyed from the neuromodulation lead 12(2), thereby decreasing the excitability of the right DR nerve fiber that may otherwise be inadvertently be stimulated by the conveyance of the electrical energy from the neuromodulation lead 12(1). For example, electrode E2 on the neuromodulation lead 12(1) can be selected to convey the electrical energy to stimulate the left DR nerve fiber, while optical element L2 on the neuromodulation lead 12(2) can be selected to convey the laser energy to reduce the excitability of the right DR nerve fiber.

In another example illustrated in FIG. 24, two hybrid neurostimulation leads 12(3) may be epidurally located above two respective columns of DR nerve fibers. Electrical energy having a relatively low frequency (e.g., 1 Hz-500 Hz) can be conveyed from a selected one of the electrodes E1-E4 from each of the neuromodulation lead 12(3), thereby increasing the excitability and stimulating a pair of left and right DR nerve fibers (which may be at the same vertebral level) and treating chronic pain in an anatomical region innervated by the left and right DR nerve fibers. However, because electrical energy is relatively unfocused, the DR nerve fibers above and below the pair of DR nerve fibers that are stimulated, may themselves be inadvertently stimulated, thereby causing a side-effect (e.g., motor movement or painful sensations). Thus, laser energy having a relatively low wavelength (e.g., 600 nm-1200 nm) can be conveyed from a selected one or ones of the optical elements L1-L4 from each of the neuromodulation leads 12(3), thereby decreasing the excitability of the DR nerve fibers that may otherwise be inadvertently be stimulated by the conveyance of the electrical energy from the neuromodulation leads 12(3). For example, electrode E2 on each of the neuromodulation leads 12(3) can be selected to convey the electrical energy to stimulate the adjacent DR nerve fibers, while optical elements L1 and L2 on each of the neuromodulation leads 12(3) can be selected to convey the laser energy to reduce the excitability of the surrounding DR nerve fibers.

In still another example illustrated in FIG. 25, one electrical neuromodulation lead 12(1) may be epidurally located above the DC nerve fibers, and two optical neuromodulation leads 12(2) may be epidurally located above the left and right DR nerve fibers. Electrical energy having a relatively low frequency (e.g., 1 Hz-500 Hz) can be conveyed from the neuromodulation lead 12(1), thereby increasing the excitability and stimulating the DC nerve fibers, and treating chronic pain in an anatomical region innervated by the DC nerve fibers. However, because electrical energy is relatively unfocused, the left and right DR nerve fibers may be inadvertently stimulated, thereby causing a side-effect (e.g., motor movement or painful sensations). Thus, laser energy having a relatively low wavelength (e.g., 600 nm-1200 nm) can be conveyed from the neuromodulation leads 12(2), thereby decreasing the excitability of the left and right DR nerve fibers that may otherwise be inadvertently be stimulated by the conveyance of the electrical energy from the neuromodulation lead 12(1). For example, electrode E2 on the neuromodulation lead 12(1) can be selected to convey the electrical energy to stimulate the left DR nerve fiber, while optical element L2 on each of the neuromodulation leads 12(2) can be selected to convey the laser energy to reduce the excitability of the left and right DR nerve fibers.

In yet another example illustrated in FIG. 26, two electrical neuromodulation leads 12(1) may be epidurally located above the left and right DR nerve fibers, and one optical neuromodulation leads 12(2) may be epidurally located above the DC nerve fiber. Electrical energy having a relatively low frequency (e.g., 1 Hz-500 Hz) can be conveyed from the neuromodulation lead 12(1), thereby increasing the excitability and stimulating the DR nerve fibers, and treating chronic pain in an anatomical region innervated by the DR nerve fibers. However, because electrical energy is relatively unfocused, the DC nerve fibers between the DR nerve fibers may be inadvertently stimulated, thereby causing a side-effect (e.g., paresthesia). Thus, laser energy having a relatively low wavelength (e.g., 600 nm-1200 nm) can be conveyed from the neuromodulation leads 12(2), thereby decreasing the excitability of the DC nerve fibers that may otherwise be inadvertently be stimulated by the conveyance of the electrical energy from the neuromodulation lead 12(1). For example, electrode E2 on each of the neuromodulation leads 12(1) can be selected to convey the electrical energy to stimulate the left and right DR nerve fibers, while optical element L2 on the neuromodulation lead 12(2) can be selected to convey the laser energy to reduce the excitability of the DC nerve fibers.

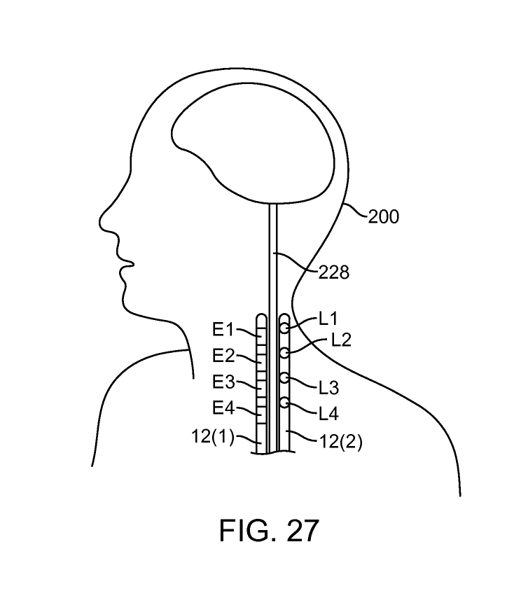

In one special case where low-level laser energy is conveyed to a first neuronal element to decrease a side-effect otherwise caused by the modulation of the first neuronal element by the electrical energy, a vagus nerve 228 of a patient 200, as shown in FIG. 27, can be modulated in order to treat any one of a variety of ailments, including heart failure, asthma, diabetes, obesity, intestinal disorder, and constipation. However, because certain nerve fibers in the vagus nerve 228 innervate different anatomical regions of the patient (e.g., heart, lung, pancreas, stomach, and intestine), only select nerve fibers in the vagus nerve 228 should be targeted for stimulation to treat a specific ailment. To this end, the electrical neuromodulation lead 12(1) (or optionally a hybrid neuromodulation lead 12(3)) is located on one lateral side of a vagus nerve 228, whereas the optical neuromodulation lead 12(2) (or optionally a hybrid neuromodulation lead 12(3)) is located on another lateral side of the vagus nerve 228.

Electrical energy having a relatively low frequency (e.g., 1 Hz-500 Hz) can be conveyed from the neuromodulation lead 12(1), thereby increasing the excitability and stimulating the nerve fibers located on the one lateral side of the vagus nerve 228, and treating ailment affecting the anatomical region innervated by these vagal nerve fibers. However, because electrical energy is relatively unfocused, the nerve fibers located on the other lateral side of the vagus nerve 228 may be inadvertently stimulated, thereby causing a side-effect. Thus, laser energy having a relatively low wavelength (e.g., 600 nm-1200 nm) can be conveyed from the neuromodulation lead 12(2), thereby decreasing the excitability of these other vagal nerve fibers that may otherwise be inadvertently be stimulated by the conveyance of the electrical energy from the neuromodulation lead 12(1). For example, electrodes E1-E4 on the neuromodulation lead 12(1) can be selected to convey the electrical energy to stimulate the targeted vagal nerve fibers, while optical elements L1-L4 on the neuromodulation lead 12(2) can be selected to convey the laser energy to reduce the excitability of the non-targeted vagal nerve fibers.

In another method, the neuronal element to which the conveyance of the electrical energy or the low-level laser energy modulates, is not associated with a side-effect resulting from the therapy, but rather the therapy itself. For example, one of the electrical energy and low-level laser energy may be used to condition a target portion of a plurality of neuronal elements by increasing their excitability, and the other of the electrical energy and low-level laser energy may be conveyed to the plurality of neuronal elements, such that the conditioned portion of the plurality of neuronal elements is stimulated without stimulating the remaining portion (i.e., the unconditioned) of the plurality of neuronal element that may otherwise cause side-effects. In this manner, more focused optical energy, although not used as the energy that actually stimulates the target neuronal elements, can be used to select the target neuronal element to be actually stimulated by the more unfocused electrical energy.

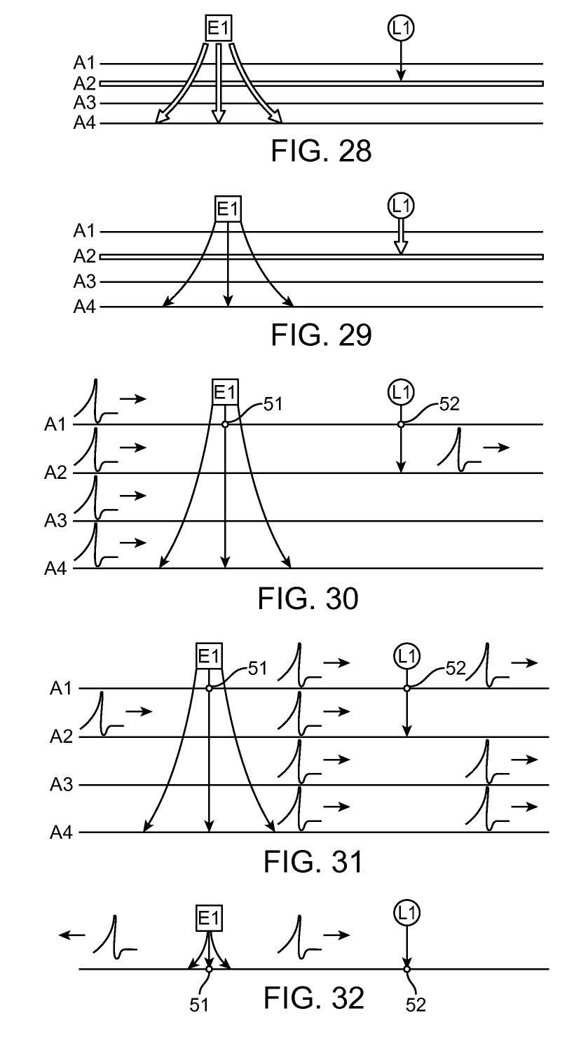

In one example illustrated in FIG. 28, a population of neural axons A1-A4 includes a target neural axon A2, the stimulation of which will result in a therapeutic effect, and non-target neural axons A1 and A3-A4, the stimulation of which will result in a side-effect. Low-level laser energy having a relatively long wavelength and small amplitude (e.g., 1400 nm-2500 nm) is conveyed from optical element L1 to the neural axon A2, thereby increasing the excitability of the neural axon A2, and conditioning it for subsequent stimulation. Electrical energy having a relatively low frequency (1 Hz-500 Hz) is conveyed from an electrode E1 to all of the neural axons A1-A4. The amplitude of the electrical energy may be relatively low, such that only the most excitable ones of the neural axons A1-A4 are stimulated. In this case, due to the conditioning laser energy, the more excitable neural axon A2 will be stimulated by the electrical energy, whereas the remaining neural axons A1 and A3-A4 will not be stimulated by the electrical energy.

In another example illustrated in FIG. 29, electrical energy having a relatively low frequency (e.g., 1 Hz-500 Hz) is conveyed from an electrode E1 to the plurality of neural axons A1-A4, thereby increasing the excitability of the neural axons A1-A4, and conditioning them for subsequent stimulation. Low-level laser energy having a relatively long wavelength (1400 nm-2500 nm) is conveyed from an optical element L1 to the neural axon A2. The amplitude of the electrical energy may be relatively low, such that the neural axons A1-A4 are not stimulated by the electrical energy. However, the addition of the focused low-level laser energy to only the conditioned neural axon A2 will stimulate it without stimulating the remaining conditioned neural axons A1 and A3-A4.

In still another method, the propagation of action potentials intrinsically generated in a population of neural axons may be limited to a targeted portion of those neural axons.

In one example illustrated in FIG. 30, intrinsically generated action potentials (either single or compound) are conveyed along a pathway that includes the neural axons A1-A4. The action potentials are generated in all four of the axons A1-A4 Electrical energy having a relatively high frequency (e.g., 1 KHz-50 KHz) is conveyed from an electrode E1 to a first target site S1 along the neural axons A1-A4, thereby decreasing the excitability of the neural axons A1-A4, and blocking the action potentials at the first target site S1 in all the neural axons A1-A4, while the low-level laser energy having a relatively long wavelength (1400 nm-2500 nm) is conveyed from a lens L1 to a second target site S2 (distal to the first target site S1) along the neural axon A2, thereby increasing the excitability of the neural axon A2 at the second target S2. As such, the action potentials on the neural axon A2 can be conveyed distally of the second target site S2.

In another example illustrated in FIG. 31, intrinsically generated action potentials (either single or compound) are conveyed along a pathway that includes the neural axons A1-A4. The action potentials are only generated in the neural axon A2. Electrical energy having a relatively low frequency (e.g., 1 Hz-500 Hz) is conveyed from an electrode E1 to a first target site S1 along the neural axons A1-A4, thereby increasing the excitability of the neural axons A1-A4, thereby evoking more action potentials in the remaining neural axons A1 and A3-A4 at the first target site S1, while low-level laser energy having a relatively short wavelength (600 nm-1200 nm) is conveyed from a lens L1 to a second target site S2 along the neural axon A2, thereby decreasing the excitability of the neural axon A2, and blocking the action potentials at the second target site S2. As such, the action potentials on the neural axons A1 and A3-A4 can be conveyed distally of the second target site S2.

In yet another method, action potentials that have been bi-directionally evoked in a neuronal element may be blocked in one direction. In one example illustrated in FIG. 32, electrical energy having a relatively low frequency (e.g., 1 Hz-500 Hz) is conveyed from an electrode E1 to a neural axon A1 at a first target site S1, thereby increasing the excitability of, and bi-directionally evoking action potentials in the, neural axon A1. Low-level laser energy having a relatively long wavelength (1400 nm-2500 nm) is conveyed from an optical element L1 to the neural axon A1 at a second target site S2, thereby decreasing the excitability of the neural axon A1, and blocking the action potentials at the second target site S2.

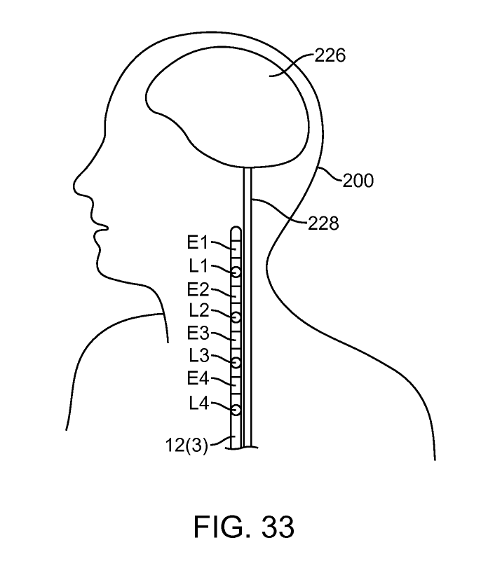

In one special case where bi-directionally evoked action potentials can be blocked in one direction, a vagus nerve 228 of a patient 200, as shown in FIG. 33, can be modulated in order to treat any one of a variety of ailments, including heart failure, asthma, diabetes, obesity, intestinal disorder, and constipation in the same manner discussed above with respect to FIG. 27. However, in this case it is desirable to block action potentials from being conveyed to the brain 226 of the patient 200 to prevent the side-effect of paresthesia. To this end, a hybrid neuromodulation lead 12(3)) can be located adjacent the vagus nerve 228. Electrical energy having a relatively low frequency (e.g., 1 Hz-500 Hz) can be conveyed from the neuromodulation lead 12(3), thereby increasing the excitability and bi-directionally evoking action potentials desired nerve fibers in the vagus nerve 228. The evoked action potentials travelling in the caudal direction will treat the ailment affecting the anatomical region innervated by these vagal nerve fibers, whereas the evoked action potentials traveling in the rostral direction will create the side-effect of paresthesia. Laser energy having a relatively low wavelength (e.g., 600 nm-1200 nm) can be conveyed from the neuromodulation lead 12(3), thereby decreasing the excitability of the vagal nerve fibers at a site rostral to the stimulation site of the electrical energy, thereby blocking the action potentials from reaching the brain 226. For example, electrode E4 on the neuromodulation lead 12(3) can be selected to convey the electrical energy to stimulate the vagal nerve fibers, while optical element L1 on the same neuromodulation lead 12(3) can be selected to convey the laser energy to block the rostrally traveling action potentials.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A method of treating a patient with an ailment using an optical element implanted within the patient, comprising:
conveying low-level laser energy having a wavelength in the range of 600 nm-2500 nm from the optical element to a neuronal element of the patient, thereby modulating the neuronal element to treat the ailment.

2. The method of claim 1, wherein the low-level laser energy decreases the excitability of the neuronal element.

3. The method of claim 1, wherein the low-level laser energy increases the excitability of the neuronal element.

4. The method of claim 1, wherein the wavelength of the low-level laser energy is in the range of 600 nm-1200 nm.

5. The method of claim 1, wherein the wavelength of the low-level laser energy is in the range of 600 nm-900 nm.

6. The method of claim 1, wherein the wavelength of the low-level laser energy is in the range of 1400-2500 nm.

7. The method of claim 1, wherein the neuronal element is a central nervous system (CNS) neuronal element.

8. The method of claim 7, wherein the CNS neuronal element is a brain structure.

9. The method of claim 8, wherein the brain structure is one of a thalamus, subthalamic nucleus (STN), globus pallidus, subgenual cingulate cortex, rostral cingulate cortex, ventral striatum, nucleus accumbens, inferior thalamic peduncle, lateral habernula, ventromedial hypothalamus, ventrolateral thalamus, zona incerta, posteroventral globus pallidus pars ilnternus, periventricular gray (PVG), periaqueductal gray (PAG), cerebellum, centromedian nucleus of thalamus, anterior nucleus of thalamus, caudate nucleus, mesial temporal lobe, ventral capsule (VC), ventral striatum (VS), pars interna of the globus internal, and anterior limb of internal capsule.

10. The method of claim 8, wherein the ailment is one or more of Parkinson's disease, depression, addiction, obesity, tremor, dystonia, pain, epilepsy, obsessive compulsive disorder, and Tourette's syndrome.

11. The method of claim 7, wherein the CNS neuronal element is a spinal cord.

12. The method of claim 11, wherein the CNS neuronal element is one of a dorsal column, ventral column, lateral column, and dorsal horn.

13. The method of claim 11, wherein the ailment is chronic pain.

14. The method of claim 1, wherein the neuronal element is a peripheral nervous system (PNS) neuronal element.

15. The method of claim 14, wherein the PNS neuronal element is a spinal nerve.

16. The method of claim 14, wherein the PNS neuronal element is a dorsal root ganglion (DRG).

17. The method of claim 14, wherein the ailment is DRG hyperactive-induced pain.

18. The method of claim 1, wherein the optical element is epidurally implanted within the patient.
